SEQUENCE LISTING (22 sequences)

SEQ ID NO: 1 - DNA, 181 bp, Artificial Sequence
  Synthetic tac promoter
ggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacag

SEQ ID NO: 2 - DNA, 535 bp, Artificial Sequence
  Synthetic gap promoter
aacttcggcggcgcccgagcgtgaacagcacgggctgaccaacctgtgcgcgcgcggcggctacgtcctggcggaagccgaagggacgcggcaggtcacgctggtcgccacggggcacgaggcgatactggcgctggcggcacgcaaactgttgaaggacgcaggggttgcggcggctgtcgtatcccttccatgctgggaactgttcgccgcgcaaaaaatgacgtatcgtgccgccgtgctgggaacggcaccccggatcggcattgaagccgcgtcagggtttggatgggaacgctggcttgggacagacgggctgtttgttggcattgacgggttcgggacggccgccccggaccagccggacagcgcgactgacatcacgccggaacggatctgccgcgacgcgctgcgtctggtccgtcccctgtccgataccctgactgaaccggcgggaggaaacggcgcgccgcccgggatgacatcggccgatgtcagtgtgtgaaatgtcagaccttacggagaaaataagaaa

SEQ ID NO: 3 - protein, 520 aa, Komagataeibacter xylinus
MASMDETFRTTAAPGGLLDLLVVGGGVNGTGIARDAAGRGASVLLVEQDDLASHTSSASTKLIHGGLRYLEYYEFRLVREALIEREKLLRIAPHIIWPMRFVLPYTPQARPAWMLRLGLFLYDHLAPNMTLPKCKSLDFRTSSAGQPLNGKLARGFAYSDGWVQDSRLVVLNAMDARARGADIRTRTRMVAARRVGGVWEADIENMLDGTKTTVRARVLVNAGGPWVSEVLRERAQVESTKNVRLVKGSHIVVPRLFDGPQAYILQNPDKRIVFAIPYEQKFTLIGTTDVPWTQAPGDVEISPEEISYLCESVSRYFTRPVTPADVVWSYAGVRPLYDDAAKNASAVTRDYVLDVDTQGNQAPMLSIFGGKITTYRRLAEHAIEKLQPFLPVLSAPGWTADKVLPGGDLGEGGFEGALARLRAQAPFLGDELSWRLVRNYGSRATEIVGDAHGMEDMGELFGAGLSVREVEYLIANEWAQTTQDILWRRSRLGLHVTDEDTARLEAYLKARKPGTAPTSA

SEQ ID NO: 4 - protein, 499 aa, Komagataeibacter xylinus
MNKKNRILAIDQGTTSTRSIVFDRDITAISVARIEFAQHYPSQGRVEHDPEEIWSNVLSTAREAIEKAGGPDVIAGIGITNQRETIVVWERSTGRPIHRAIVWQDRRTTPICARMHEEGLEPLVRERTGLLLDPYFSATKIAWILDNVEGARAQAEKGELACGTIDSFLLWRLTGGRVHATDTTNASRTLLFNIHTCAWDDELLALFKVPRAILPEVRTNSEVFGETTPELFGAPLKVAGMAGDQNAAMVGQACFRPGTAKATYGTGCFALLNTGTTPVMSENRMLTTIAYRIGAETTYALEGSIFVAGAAIRWLRDGLNLITHASQTDDMATRVPHSHGVYMVPGFVGLGAPHWDPDARGLICGLTLDATAAHIARAALESVAYQTMDLMDAMHEDGGCKLNALRVDGGMSVNDWFCQFLADMLLTPVERPRQVETTALGAAFLAGLATGVWESIAELEGTWTRGHLFRPTMDKAQRDTMVAGWHVAVRRTLSSTVAA

SEQ ID NO: 5 - protein, 328 aa, Komagataeibacter xylinus
MTTTRHNPYQVTDRNLALELVRVTEAAAVAASAWTGRGLKNEADGAAVEAMRRAFDTVAIDGTVVIGEGEMDEAPMLFIGEKVGSGGPGMDIAVDPLEGTNLCAKNLPNALTVVALAESGNFLHAPDIYMDKIVVGPYLPEGVVDLDSTIEANLKSLAQAKKCAVSDLMLCTLDRERHEELIARARAAGARVTLLSDGDVAAAIAACLDDSEIDIYVGSGGAPEGVLAAAAVRCVHGQMQGRLLFEDDDQVARARKMNPGADPSRKLALEDMARGDVLFSATGVTGGALLHGIRRNGIRTVTHSLVMRSKSGTIRFVEGHHDYQTKTW

SEQ ID NO: 6 - protein, 368 aa, Komagataeibacter xylinus
MTNTAHTASGRLGLRPGVVTGADYRRLVETCRDEGYALPAVNVVGTDSINAVLEAAARNRADVIIQLSNGGARFYAGEGMKDAEQARVLGAVAAARHVHTVAAAYGVCVILHTDHADRKLLPWISGLIDASEEAVKETGRPLFSSHMIDLSAEPLEDNIAECARFLRRMAPLGIGLEIELGVTGGEEDGIGHDLDDGADNAHLYTQPEDVLKAYEALSPLGFVTIAASFGNVHGVYAPGNVKLRPEILRNSQAAVAKATNLGEKPLALVFHGGSGSEQAKITEAVSYGVFKMNIDTDIQFAFASSIGHYVQEHAEAFSHQIAPSTGKPTKKLYDPRKWLRVGEQGIVARLEQSFADLGATGRSVARAV

SEQ ID NO: 7 - protein, 252 aa, Komagataeibacter xylinus
VSAEERHREITALVRTQGYVSNEDLAQRLNVAVQTIRRDVNLLARRGLVARHHGGAGLASSVENIAYSERQVLNRRAKEAIGSLAARQIPDNSSLFVSIGTTTEAFAKSLRRHKALRVITNNLHVATPLSAQTDFQVIVTGGQVRFYDGGITGSTASTFIEQYRTDFAVIGISGIEDDGTLLDFDADEISVAQAMMRNARRVYLLADQTKFGRRPMGRLGHLSHVHGFFTDRQPSEQICAMLRAHDVELHIA

SEQ ID NO: 8 - DNA, 4381 bp, Artificial Sequence
  Synthetic pMKO vector
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccaacttcggcggcgcccgagcgtgaacagcacgggctgaccaacctgtgcgcgcgcggcggctacgtcctggcggaagccgaagggacgcggcaggtcacgctggtcgccacggggcacgaggcgatactggcgctggcggcacgcaaactgttgaaggacgcaggggttgcggcggctgtcgtatcccttccatgctgggaactgttcgccgcgcaaaaaatgacgtatcgtgccgccgtgctgggaacggcaccccggatcggcattgaagccgcgtcagggtttggatgggaacgctggcttgggacagacgggctgtttgttggcattgacgggttcgggacggccgccccggaccagccggacagcgcgactgacatcacgccggaacggatctgccgcgacgcgctgcgtctggtccgtcccctgtccgataccctgactgaaccggcgggaggaaacggcgcgccgcccgggatgacatcggccgatgtcagtgtgtgaaatgtcagaccttacggagaaaataagaaaagatctcaataatattgaaaaaggaagagtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgatgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 9 - DNA, 6126 bp, Artificial Sequence
  Synthetic pJET-EX vector
gcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggccttttcatgattacgggcagatcttcgcctttgacgaatgggccgcgagcgaccagcccgacccccgccccgccacctgacaccagccattggggaggccgccatgcaaggcggcctccctgcgggaaccctgcgtcatggacaccatgctcacgacccagaccatcctctctctcctgcccgcccggtatgccgcggatgcggttgtcatcttctccttcctcatttccggctgtgcgctcgtcgcgcgcttctggcggccacccgcagccgggtcgaaatgggtggtcgtgtggacctttgtaaccgccatggcgcaactgcgtggctggagcaggccccctgacaggaaaggcgatgccacggataagaaaccgtaaagaggtttcgggtgaagcttttttttaaaagattctgaagaaaactgcctttttaacaaacagcagggcaaaaatgatgctgcgtaaacttggctgccgccctgccgaaaggcgtgcgcgccagcccatgctcacaaccatgcggggcttcatggcccgccgcgcgccacagcacctgaaccgcgatggcatcgatcccgccccgctcatgctgggcaatgatgtgctgggtgactgcacggcggcgggcataggcaaccatatccgcgccactgccgcacttgcgggctatcaggtggcgatggatacgcccgatgccgtgcggttctacgcgctttccaccggttatgtgcccggcaacccggccaccgatcatggcggtgtggaagtggatgtgctgagcaggtcgactctagatatctttctagaagatctcctacaatattctcagctgccatggaaaatcgatgttcttcttttattctctcaagattttcaggctgtatattaaaacttatattaagaactatgctaaccacctcatcaggaaccgttgtaggtggcgtgggttttcttggcaatcgactctcatgaaaactacgagctaaatattcaatatgttcctcttgaccaactttattctgcattttttttgaacgaggtttagagcaagcttcaggaaactgagacaggaattttattaaaaatttaaattttgaagaaagttcagggttaatagcatccattttttgctttgcaagttcctcagcattcttaacaaaagacgtctcttttgacatgtttaaagtttaaacctcctgtgtgaaattattatccgctcataattccacacattatacgagccggaagcataaagtgtaaagcctggggtgcctaatgagtgagctaactcacattaattgcgttgcgctcactgccaattgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccgcccctgcagccgaattatattatttttgccaaataatttttaacaaaagctctgaagtcttcttcatttaaattcttagatgatacttcatctggaaaattgtcccaattagtagcatcacgctgtgagtaagttctaaaccatttttttattgttgtattatctctaatcttactactcgatgagttttcggtattatctctatttttaacttggagcaggttccattcattgtttttttcatcatagtgaataaaatcaactgctttaacacttgtgcctgaacaccatatccatccggcgtaatacgactcactatagggagagcggccgccagatcttccggatggctcgagtttttcagcaagtatagggcgaattcgtagcgcaggaagaaagccaccagcgcccacaggggcagggccatgagcaggctgaaaaagatgccactcgcggcggaataccggcggcgggcagggacagtcactcgctgggcagcaggctgggaaaccgtctgtgtcagggcgataccatcaaacgacatgcgcttagggccttagaaactgaaggaaaggggaaaagcacccccaattgtggagtagcaccacaatcctgccttaaaaataacacgatctgctgtcaatcacttttaattaaactgccatcattatcgctgcctgcatctgcgcagggggctataaaatctggcattaacagacacttccataaaagttacgggttccgcccctgcccggcagcagccagcgcagtatggctttccgtgccatagggtgcggacccgtaccccgaaatgcatctgttcggccacgattcccgcccagcgggcttgtggcctgcaaccggggttccatctgccgcagggccgcgcgctgcgccggggcaatggcccgatcgggtcaagccggtacgcgacggcaggcgtgagaaaaatctgttcgtatcagccagtcctgaaatttcacgggcgggcgcatgctttcttttgctgcctgcatgggcgcgccctatatttcatcttgtcaggagcgaaaagacaacgcgattaccctgaccgcgaaagtataatggcataattcatgcattatacagaacagatacctgcatataaatagatcagggctgtcatcatgccctgtcgagaggatcagatcggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacatagatctcccgggtaccgagctctctagaaagaaggagggacgagctattgatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacagggacgagctattgattgggtaccgagctcgaattcgtacccggggatcctcta

SEQ ID NO: 10 - DNA, 1689 bp, Artificial Sequence
  Synthetic Kan gene
aacttcggcggcgcccgagcgtgaacagcacgggctgaccaacctgtgcgcgcgcggcggctacgtcctggcggaagccgaagggacgcggcaggtcacgctggtcgccacggggcacgaggcgatactggcgctggcggcacgcaaactgttgaaggacgcaggggttgcggcggctgtcgtatcccttccatgctgggaactgttcgccgcgcaaaaaatgacgtatcgtgccgccgtgctgggaacggcaccccggatcggcattgaagccgcgtcagggtttggatgggaacgctggcttgggacagacgggctgtttgttggcattgacgggttcgggacggccgccccggaccagccggacagcgcgactgacatcacgccggaacggatctgccgcgacgcgctgcgtctggtccgtcccctgtccgataccctgactgaaccggcgggaggaaacggcgcgccgcccgggatgacatcggccgatgtcagtgtgtgaaatgtcagaccttacggagaaaataagaaaagatctcaataatattgaaaaaggaagagtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgatgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgc

SEQ ID NO: 11 - DNA, 40 bp, Artificial Sequence
  Synthetic primer
gcaggtcgactctagaggtacagcgtatagaccagcattg

SEQ ID NO: 12 - DNA, 35 bp, Artificial Sequence
  Synthetic primer
cgccgaagttcctcctgatacgtatgttcgtaacc

SEQ ID NO: 13 - DNA, 34 bp, Artificial Sequence
  Synthetic primer
ggtaccgagcatggcgtccatggacgaaacattc

SEQ ID NO: 14 - DNA, 39 bp, Artificial Sequence
  Synthetic primer
gacggccagtgaattccaccacgatatggctgcccttga

SEQ ID NO: 15 - DNA, 34 bp, Artificial Sequence
  Synthetic primer
tatcaggaggaacttcggcggcgcccgagcgtga

SEQ ID NO: 16 - DNA, 35 bp, Artificial Sequence
  Synthetic primer
tgcacagccagaattcgagctcggtacccggggat

SEQ ID NO: 17 - DNA, 38 bp, Artificial Sequence
  Synthetic primer
gctcgaattctggctgtgcaggtcgtaaatcactgcat

SEQ ID NO: 18 - DNA, 35 bp, Artificial Sequence
  Synthetic primer
tggacgccatgctcggtacccaatcaatagctcgt

SEQ ID NO: 19 - DNA, 25 bp, Artificial Sequence
  Synthetic primer
ggcatgcagcggcgtaatgccttcg

SEQ ID NO: 20 - DNA, 25 bp, Artificial Sequence
  Synthetic primer
aatggcgaagacgatgcgcttgtcc

SEQ ID NO: 21 - DNA, 284 bp, Komagataeibacter xylinus
gaaatttctccaacattccggattcgggggcaggtgatgttgtggtggtgatggaaggtaagggatggggttgtggaacgagatgcgcgatttcgcacatgcacaaaaaccgctccggcatataaattactcggaatggcgaatttatggctaatgccgagctctatcatgcatggaaaaagaaagaattattgagacgtgcggcatgcagaaatgtgcgcgtgccctcttgaatcgatcagaatgttcggttacgaacatacgtatcaggaggaacattgact

SEQ ID NO: 22 - DNA, 759 bp, Komagataeibacter xylinus
gtgtcagcagaagaacgtcaccgggaaattaccgcgctggtgcgtacccagggctatgtctccaacgaggatctggcccagaggctgaatgttgcggtccagaccatccgccgtgatgtcaacctccttgcccgtcgcgggttggtggcgcggcatcatggcggggcgggtctggcctcgtcggtcgagaatatcgcctattccgagcggcaggtgctcaaccggcgggccaaggaagccattggcagccttgcggcccgccagatccctgacaattcatcgctttttgtcagcatcggcaccacgaccgaagcctttgccaaatccttgcggcggcacaaggcgctgcgggtcattaccaacaatctgcatgtggccaccccgctttccgcccagaccgattttcaggtgatcgtgacgggggggcaggtgcggttttatgatggtggcattaccggctccaccgccagcaccttcatcgagcagtatcgcaccgattttgccgtaattggcatcagcggcatcgaagatgatggcacgctgcttgatttcgatgccgatgaaatcagcgtggcccaggccatgatgcgcaatgcaaggcgtgtctacctgttggccgaccagaccaaattcggccgccggcccatgggccggctcgggcacctttcgcatgtgcatggcttttttaccgaccggcagccatccgagcagatctgcgccatgctgcgtgctcatgacgtggaactgcatatcgcctga

(19) United States
(12) Patent Application Publication
(10) Pub. No.: US 2019/0144904 A1
(43) Pub. Date: May 16, 2019

(54) MICROORGANISM HAVING ENHANCED CELLULOSE PRODUCTIVITY, METHOD OF PRODUCING CELLULOSE BY USING THE SAME, AND METHOD OF PRODUCING THE MICROORGANISM

(71) Applicant: Samsung Electronics Co., Ltd., Suwon-si (KR)

(72) Inventors: Wooyong Shim, Suwon-si (KR); Hongsoon Rhee, Suwon-si (KR); Sunghaeng Lee, Seoul (KR); Jinhwan Park, Suwon-si (KR)

(21) Appl. No.: 16/191,133

(22) Filed: Nov. 14, 2018

(30) Foreign Application Priority Data
Nov. 15, 2017 (KR) 10-2017-0152498

(51) Int. Cl. C12P 19/04; C12N 15/74; C12N 9/04; C12N 9/12; C12N 9/16; C12N 9/88

(52) U.S. Cl. CPC C12P 19/04; C12N 15/74; C12N 9/0006; C12Y 101/01008; C12Y 401/02013; C12Y 207/0103; C12N 9/16; C12Y 301/03011; C12N 9/88; C12N 9/1205

(57) ABSTRACT

Provided are a microorganism having enhanced cellulose productivity, a method of producing cellulose by using the microorganism, and a method of producing the microorganism.

### CROSS-REFERENCE TO RELATED APPLICATION

This application claims the benefit of Korean Patent Application No. 10-2017-0152498, filed on Nov. 15, 2017, in the Korean Intellectual Property Office, the entire disclosure of which is hereby incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 37,529 Byte ASCII (Text) file named “740189_ST25.TXT”, created on Nov. 14, 2018.

### BACKGROUND

### 1. Field

The present disclosure relates to a recombinant microorganism having enhanced cellulose productivity, a method of producing cellulose by using the same, and a method of producing the microorganism.

### 2. Description of the Related Art

In cellulose produced by microbial culture, glucose is present in a primary structure of β-1,4 glucan and forms a network of multiple strands of fibrils. This cellulose is referred to as “bio-cellulose” or “microbial cellulose.”

Unlike plant cellulose, microbial cellulose is pure cellulose in which lignin or hemicellulose is not present. Microbial cellulose has a fiber width of 100 nm or less, and has desirable wettability, absorbency, high strength, high resilience, and high heat resistance characteristics. Due to these properties, microbial cellulose is useful in various industries, such as cosmetics, medical, dietary fiber, acoustic diaphragm, and functional film.

Therefore, to meet the demands for microbial cellulose, there is a need to produce microorganisms having enhanced cellulose productivity. This invention provides such a microorganism.

### SUMMARY

An aspect of the invention provides a microorganism comprising a genetic modification that enhances an expression of at least one gene selected from a gene encoding glycerol-3-phosphate dehydrogenase (glpD), a gene encoding glycerol kinase (glpK), a gene encoding fructose-1,6-bisphosphatase (glpX), and a gene encoding fructose-bisphosphate aldolase (FBA) 3, wherein expression of the at least one gene is regulated by a glycerol operon.

Another aspect of the invention provides methods of producing cellulose by using the microorganism comprising said genetic modification.

Another aspect of the invention provides methods of producing the microorganism comprising said genetic modification.

### BRIEF DESCRIPTION OF THE DRAWING

These and/or other aspects will become apparent and more appreciated from the following description of the embodiments, taken in conjunction with the drawings in which:

FIG. 1 schematically illustrates a DNA construct for replacing a DNA glycerol operon promoter through homologous recombination.

### DETAILED DESCRIPTION

Additional aspects of the present invention will be set forth in part in the description which follows and, in part, will be apparent from the description, or may be learned by practice of the presented embodiments.

The term “increase in activity” or “increased activity,” or a similar term, as used herein, may indicate a detectable increase in the activity of a cell, protein, or enzyme. The term “increase in activity” or “increased activity” or the like refers to the activity of a cell, protein, or enzyme that is modified (for example, genetically engineered) to a level that is higher than the level of a comparable cell, protein, or enzyme of the same type, such as a cell, protein, or enzyme that does not have the given genetic modification (for example, native or “wild-type” cell, protein, or enzyme). For example, the activity of the modified or engineered cell, protein, or enzyme may be greater than the activity of the same type of cell, protein, or enzyme that has not been engineered, such as a wild-type cell, protein, or enzyme, by about 5% or more, about 10% or more, about 15% or more, about 20% or more, about 30% or more, about 50% or more, about 60% or more, about 70% or more, or about 100% or more. Cells including proteins or enzymes having enhanced activities may be identified by using any method known in the art.

The increase in activity of an enzyme or a polypeptide may be achieved by increased expression of the enzyme or polypeptide, and/or increased specific activity thereof. The enhanced expression may be achieved by the introduction of an enzyme or polypeptide, or a polynucleotide encoding the enzyme or polypeptide, into a cell. The enhanced expression may also be achieved by an increase in the copy number of a polynucleotide encoding an enzyme or polypeptide or by a mutation in a regulatory region of the polynucleotide that increases expression. A microorganism into which the polynucleotide encoding the enzyme is introduced may be a microorganism that endogenously contains the gene or may be a microorganism that does not endogenously contain the gene. The gene may be operably linked to a regulatory sequence enabling its expression, for example, a promoter, an operator, an enhancer, a polyadenylation site, or a combination thereof. An endogenous gene refers to a gene that is present in the genetic material contained within a microorganism. An exogenous gene refers to a gene that is introduced into the cell from the outside. The introduced gene may be homologous or heterologous with respect to the host cell to be introduced. The term “heterologous” means that the gene is “foreign”, or not “native” to the species.

The “copy number increase” of a gene may be due to the introduction of an exogenous gene or amplification of an endogenous gene, and includes, for instance, the introduction of an exogenous gene into a microorganism that did not previously include a copy of the gene. The introduction of the gene may be mediated by a vehicle, such as a vector. The introduction may be a transient introduction in which the gene is not integrated into the genome, or the introduction may be an introduction where the gene is inserted into the genome. The introduction may be performed as follows: for example, a vector into which a polynucleotide encoding a target polypeptide has been inserted is introduced into a cell, and then, the vector is replicated in the cell or the polynucleotide is integrated into the genome. The introduction may be made by a Clustered Regularly Interspaced Short Palindromic Repeats (CRISPR)-binding system.

The introduction of the gene may be carried out by known methods such as transformation, transfection, electroporation, and the like. The gene may be introduced via a vehicle or introduced alone. The term “vehicle” used herein includes nucleic acid molecules capable of transferring other nucleic acids to which they are linked. The vehicle may be a vector, a cassette, or other a nucleic acid construct suitable for delivery of a gene. The vector may be, for example, a plasmid (e.g., plasmid expression vector). Plasmids include circular, double-stranded DNA loops to which additional DNA may be ligated. The vector may also be a virus-derived vector (e.g., virus expression vector), for example, a replication-defective retrovirus, an adenovirus, an adeno-associated virus, or a combination thereof.

The genetic engineering used herein may be performed by any molecular biological method known in the art.

The term “parent cell” refers to a cell that does not have the particular genetic modification as compared to a given modified microorganism, but is otherwise the same type of cell as the modified microorganism. Accordingly, the parental cell may be a starting material for producing a genetically engineered microorganism containing a given modification (e.g., a modification that enhances activity of a protein, such as one of the genetic modifications described herein). The parent cell includes but is not limited to a “wild-type” cell. The same comparison applies to other genetic modifications.

The “gene” used herein refers to a nucleic acid fragment encoding a particular protein, and may include or may not include a regulatory sequence of a 5′-non coding sequence and/or a 3′-non coding sequence.

The term “sequence identity” of a polynucleotide sequence or polypeptide sequence as used herein refers to the degree of similarity between corresponding nucleotide or amino acid sequences measured after the sequences are optimally aligned. In one or more embodiments, a percentage of the sequence identity may be calculated by comparing two optimally aligned corresponding sequences in an entire comparable region, determining the number of locations where an amino acid residue or a nucleotide is identical in the two sequences to obtain the number of matched locations, dividing the number of the matched locations by the total number of all locations within a comparable range (that is, a range size), and multiplying the result by 100 to obtain a percentage of the sequence identity. The percentage of the sequence identity may be determined by using a known sequence comparison program. Examples of the program include BLASTN(NCBI), BLASTP(NCBI), CLC Main Workbench (CLC bio), and MegAlign™ (DNASTAR Inc). Unless otherwise stated herein, the selection of the parameters used to execute the program may be as follows: Ktuple=2, Gap Penalty=4, and Gap length penalty=12.

In identifying a polypeptide or polynucleotide of various species which has identical or similar function or identical or similar activity, various levels of sequence identity may be available therein. For example, the sequence identity may include 50% or more, 55% or more, 60% or more, 65% or more, 70% or more, 75% or more, 80% or more, 85% or more, 90% or more, 95% or more, 96% or more, 97% or more, 98% or more, 99% or more, 100%, etc.

The term “genetic modification” used herein includes artificial change in the constitution or structure of the genetic material of a cell.

In the present specification, % generally represents w/w %, unless otherwise stated.

One aspect of the present invention provides a recombinant microorganism including a genetic modification that increases the expression of at least one gene selected from the following: a gene encoding glycerol-3-phosphate dehydrogenase (glpD), a gene encoding glycerol kinase (glpK), a gene encoding fructose-1,6-bisphosphatase (glpX), and a gene encoding bisphosphate aldolase (FBA) 3.

In one or more embodiments of the present invention, expression of the at least one gene may be regulated by a glycerol operon. The term “operon” used herein refers to a functional unit of genomic DNA, including a cluster of genes under the control of a single promoter. In one embodiment of the present invention, the genes are transcribed together into mRNA and translated together in the cytoplasm. In a further aspect of the invention, the genes are trans-spliced into monocistronic mRNA to be translated separately, that is, the genes are trans-spliced into multiple-strands of mRNAs, each encoding a single gene product. The glycerol operon may be from, for example, the species Komagataeibacter xylinum, and may include a gene encoding glycerol-3-phosphate dehydrogenase (glpD), a gene encoding glycerol kinase (glpK), a gene encoding fructose-1,6-bisphosphatase (glpX), a gene encoding fructose-bisphosphate aldolase (FBA) 3, and a gene encoding glycerol-3-phosphate repressor. The glycerol operon is a glycerol-inducible operon whose expression is induced in the presence of glycerol.

The glycerol-3-phosphate dehydrogenase (glpD) may catalyze the reversible conversion of dihydroxyacetone phosphate (DHAP) to glycerol-3-phosphate (G3P). The glpD may belong to EC 1.1.5.3, EC 1.1.1.94, or EC 1.1.1.8.

The glycerol kinase (glpK) may catalyze the reversible conversion of ATP+glycerol to ADP+glycerol-3-phosphate. The glpK may belong to EC 2.7.1.30.

The fructose-1,6-bisphosphatase (glpX) may catalyze the reversible conversion of fructose 1,6-bisphosphate+H₂O to fructose 6-phosphate+phosphate. The glpX may belong to EC 3.1.3.11.

The fructose bisphosphate aldolase (FBA) may catalyze the reaction of fructose-1,6-bisphosphate (FBP)⇔dihydroxyacetone (DHAP)+glyceraldehyde 3-phosphate (G3P). The fructose bisphosphate aldolase may belong to EC 4.1.2.13. The fructose bisphosphate aldolase may be exogenous or endogenous. The fructose bisphosphate aldolase may be in the form of a monomer consisting of a single polypeptide. The fructose bisphosphate aldolase may be selected from a fructose bisphosphate aldolase derived from the genus Gluconacetobacter, the genus Bacillus, the genus Mycobacterium, the genus Zymomonas, the genus Vibrio, and the genus Escherichia. The FBA may be FBA3. The FBA3 may facilitate the reaction of dihydroxyacetone (DHAP)+glyceraldehyde 3-phosphate (G3P)->fructose-1,6-bisphosphate (FBP) to predominate over the reverse reaction thereof.

The genetic modification may be a modification of the glycerol operon. The genetic modification may lead to an increase in the expression of the glycerol operon. The genetic modification may include at least one modification selected from (i) a disruptive mutation of a regulatory element of the glycerol operon, and/or (ii) the substitution of an operator binding site or native promoter with a constitutive promoter. For example, the native promoter may have a nucleotide sequence of SEQ ID NO: 21, and the regulatory element of the glycerol operon may be located within the nucleotide sequence of SEQ ID NO: 21. The constitutive promoter, as used herein, may be a promoter having an activity of inducing expression even in the absence of glycerol. The disruptive mutation may be attenuation or inactivation of a glycerol-3-phosphate repressor, by which the expression of the glycerol-3-phosphate repressor is reduced or prevented. An example of a repressor may be that encoded by a nucleotide sequence of SEQ ID NO: 22. The attenuation or inactivation includes deletion or inactivation of a glycerol-3-phosphate repressor gene, or the inactivation of a regulatory sequence thereof.

The substitution may be the substitution of a promoter of the glycerol operon with a constitutive promoter. The constitutive promoter may include one or more selected from a tac promoter (SEQ ID NO: 1) and a gap promoter (SEQ ID NO: 2). The disruptive mutation or the substitution may be performed by known methods, such as homologous recombination, position-directed mutagenesis, CAS, and the like.

The genetic modification may increase the expression of a gene encoding fructose-1,6-bisphosphatase (glpX) and a gene encoding fructose-bisphosphate aldolase (FBA) 3.

The genetic modification may also increase the copy number of one or more of the genes. The genetic modification may increase the copy numbers of one or more genes selected from the group consisting of a gene encoding fructose-1,6-bisphosphatase (glpX) a gene encoding fructose-bisphosphate aldolase (FBA) 3, a gene encoding glycerol-3-phosphate dehydrogenase (GlpD), and a gene encoding glycerol kinase (glpK). For instance, the genetic modification may include introducing one or more exogenous polynucleotides encoding fructose-1,6-bisphosphatase (glpX), glycerol-3-phosphate dehydrogenase (GlpD), and/or fructose-bisphosphate aldolase (FBA) 3.

In one embodiment, the glycerol-3-phosphate dehydrogenase (GlpD), the glycerol kinase (glpK), the fructose-1,6-bisphosphatase (glpX), the fructose-bisphosphate aldolase (FBA) 3, and the glycerol-phosphate regulon repressor (glpR) each have a sequence identity of 85% or more with the amino acid sequences of SEQ ID NOS: 3, 4, 5, 6, and 7, respectively.

The microorganism may belong to the genus Komagataeibacter, the genus Gluconacetobacter, or the genus Acetobacter. The microorganism may have cellulose productivity. The recombinant microorganism may have enhanced cellulose productivity compared to a parent strain thereof.

The microorganism belonging to the genus Komagataeibacter may be K. xylinus, K. europaeus, K. hansenii, K. intermedius, or K. kakiaceti. The microorganism may be K. xylinus.

The microorganism belonging to the genus Gluconacetobacter may be G. aggeris, G. asukensis, G. azotocaptans, G. diazotrophicus, G. entanii, G. europaeus, G. hansenii, G. intermedius, G. johannae, G. kakiaceti, G. kombuchae, G. liquefaciens, G. maltaceti, G. medellinensis, G. nataicola, G. oboediens, G. rhaeticus, G. sacchari, G. saccharivorans, G. sucrofermentans, G. swingsii, G. takamatsuzukensis, G. tumulicola, G. tumulisoli, or G. xylinus.

The microorganism belonging to the genus Acetobacter may be A. aceti, A. cerevisiae, A. cibinongensis, A. estunensis, A. fabarum, A. farinalis, A. indonesiensis, A. lambici, A. liquefaciens, A. lovaniensis, A. malorum, A. musti, A. nitrogenifigens, A. oeni, A. okinawensis, A. orientalis, A. orleanensis, A. papayae, A. pasteurianus, A. peroxydans, A. persici, A. pomorum, A. senegalensis, A. sicerae, A. suratthaniensis, A. syzygii, A. thailandicus, A. tropicalis, or A. xylinus.

The microorganism may have one or more genetic modifications, in addition to a genetic modification that increases the expression of a gene encoding fructose-bisphosphate aldolase (FBA) 3.

Another aspect of the present disclosure provides a method of producing cellulose, the method including: culturing the recombinant microorganism in a medium to produce cellulose; and collecting the cellulose from the culture.

The culturing may be carried out in a medium containing a carbon source, for example, glucose. The medium used for culturing the microorganism may be any conventional medium suitable for growth of host cells, such as a minimal or complex medium containing suitable supplements. Suitable media are available from commercial vendors or may be prepared according to known manufacturing methods.

The medium may be a medium that satisfies the requirements of a specific microorganism according to a selected product of the culture. The medium may be a medium selected from carbon sources, nitrogen sources, salts, trace elements, or combinations thereof. The medium may include 0.5% to 3% (v/v) ethanol.

The conditions for culturing may be appropriately adjusted to be suitable for the production of the selected product, for example, cellulose. The culturing may be carried out under aerobic conditions for cell proliferation. The culturing may be static culturing, i.e., culturing without stirring. The culturing may be culturing that is performed when the concentration of the microorganism is low. The concentration of the microorganism may be in such a range that the secretion of cellulose is not affected.

The term “culture condition” refers to conditions for culturing microorganisms. The culture condition may be, for example, a carbon source, a nitrogen source, or an oxygen condition, each used by the microorganism. The carbon source may include monosaccharides, disaccharides, or polysaccharides. The carbon source may include, as an assimilable sugar, glucose, fructose, mannose, or galactose. The nitrogen source may be an organic nitrogen compound or an inorganic nitrogen compound. The nitrogen source may be an amino acid, an amide, an amine, a nitrate salt, or an ammonium salt. The oxygen condition for culturing a microorganism includes aerobic conditions of normal oxygen partial pressure, hypoxic conditions containing 0.1% to 10% oxygen in the atmosphere, and anaerobic conditions without oxygen. Metabolic pathways may be modified to accommodate the carbon and nitrogen sources available to microorganisms.

In one embodiment, the medium may include at least one of glucose and glycerol. The combined amount of glucose and glycerol may be in an amount of 20 g/L medium, for example, greater than 0 g/L medium to 20 g/L medium, greater than 0 g/L medium to 17 g/L medium, greater than 0 g/L medium to 15 g/L medium, greater than 0 g/L medium to 13 g/L medium, greater than 0 g/L medium to 11 g/L medium, 3 g/L medium to 20 g/L medium, 5 g/L medium to 17 g/L medium, 7 g/L medium to 15 g/L medium, 10 g/L medium to 20 g/L medium, or 5 g/L medium to 20 g/L medium.

According to some embodiments, the modification to the microorganism allows for the production of CNF in a medium that does not include glycerol. Thus, in one embodiment, the culture medium does not include glycerol. In a further embodiment, the medium does not include glycreol and may include glucose in an amount of up to 20 g/L medium, for example, greater than 0 g/L medium to 20 g/L medium, greater than 0 g/L medium to 17 g/L medium, greater than 0 g/L medium to 15 g/L medium, greater than 0 g/L medium to 13 g/L medium, greater than 0 g/L medium to 11 g/L medium, 3 g/L medium to 20 g/L medium, 5 g/L medium to 17 g/L medium, 7 g/L medium to 15 g/L medium, 10 g/L medium to 20 g/L medium, or 5 g/L medium to 20 g/L medium.

In one embodiment of the present invention, the method includes the collecting of the cellulose from the culture. The collecting may be performed by, for example, obtaining a cellulose pellicle formed on the top of the medium. The cellulose pellicle may be obtained by physically separating or removing the medium. The separation may allow the cellulose pellicle to be obtained while retaining the shape of the cellulose pellicle.

Another aspect of the present invention provides a method of producing a microorganism having enhanced cellulose productivity, the method including introducing, into a microorganism, a genetic modification that increases the expression of at least one gene selected from a gene encoding glycerol-3-phosphate dehydrogenase (glpD), a gene encoding glycerol kinase (glpK), a gene encoding fructose-1,6-bisphosphatase (glpX), and a gene encoding fructose-bisphosphate aldolase (FBA) 3, wherein expression of the at least one gene is regulated by a glycerol operon, and the microorganism belongs to the genus Komagataeibacter, the genus Gluconacetobacter, or the genus Acetobacter.

The method may be a method of producing a microorganism having enhanced cellulose productivity, the method including introducing the at least one gene into a microorganism belonging to the genus Komagataeibacter, the genus Gluconacetobacter, or the genus Acetobacter. The gene may be introduced into the microorganism via a vehicle containing the gene. In this method, the genetic modification may include amplifying the gene, engineering a regulatory sequence of the gene, or engineering the sequence of the gene itself. The engineering may be insertion, substitution, conversion or addition of nucleotides.

In one aspect of the present invention, the genetic modification may include at least one of (i) a disruptive mutation of a regulatory element of the glycerol operon, and/or (ii) substitution of an operator binding site or native promoter with a constitutive promoter. The disruptive mutation may be attenuation or inactivation of the glycerol-3-phosphate repressor.

The introduction of the genetic modification may include introducing, into a microorganism, at least one gene selected from a gene encoding glycerol-3-phosphate dehydrogenase (glpD), a gene encoding glycerol kinase (glpK), a gene encoding fructose-1,6-bisphosphatase (glpX), and a gene encoding fructose-bisphosphate aldolase (FBA) 3.

The method may further include introducing, into the microorganism, a genetic modification selected from a genetic modification for enhancing the activity of phosphoglucomutase, which catalyzes the conversion of glucose-6-phosphate to glucose-1-phosphate; a genetic modification for enhancing the activity of UTP-glucose-1-phosphate uridylyltransferase, which catalyzes the conversion of glucose-1-phosphate to UDP-glucose; and a genetic modification for enhancing the activity of cellulose synthase, which catalyzes the conversion of UDP-glucose to cellulose.

Another aspect of the invention provides a recombinant microorganism having enhanced cellulose productivity as compared to a parent cell. The recombinant microorganism produces cellulose with high efficiency.

Another embodiment of the invention provides a method of producing cellulose. According to the method, cellulose may be efficiently produced.

Another aspect of an embodiment provides a method of producing a microorganism with enhanced cellulose productivity. According to the method, a microorganism with enhanced cellulose productivity may be efficiently produced.

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects.

Hereinafter, the present disclosure will be described in more detail with reference to examples. However, these examples are for illustrative purposes only, and the scope of the present disclosure is not limited to these examples.

### Example 1. Production of K. xylinus with Constitutive Promoter and Production of Cellulose

In this example, the natural promoter of a glycerol operon was substituted with a constitutive promoter in K. xylinus DSM2325 (DSM, Germany), thereby increasing expression of all of the genes under control of the operon including genes encoding glycerol-3-phosphate dehydrogenase (glpD), glycerol kinase (glpK), fructose-1,6-bisphosphatase (glpX), and fructose-bisphosphate aldolase (FBA) 3. The substitution was performed by homologous recombination. The yield of cellulose was confirmed by examining the obtained recombinant microorganism. The constitutive promoter was a Tac promoter. In general, the constitutive promoter may be any natural or synthetic promoter.

1. Production of K. xylinus with Constitutive Promoter

The glycerol operon promoter of the K. xylinus strain DSM2325 was substituted with the Tac promoter. The substitution process is as follows.

(A) Preparation of DNA Construct for the Substitution of Glycerol Operon Promoter

Using the genomic DNA of the microorganism as a template, 0.8 kb (left arm) and 0.7 kb (right arm) of a product was obtained by using glycerol operon_left forward and reverse primers (SEQ ID NOS: 11 and 12) and glycerol operon_right forward and reverse primers (SEQ ID NOS: 13 and 14).

1.7 kb of kanamycin resistance gene (Kan) product was obtained by using the pMKO vector (SEQ ID NO: 8) as a template and forward and reverse primers (SEQ ID NOS: 15 and 16). 0.3 kb of a tac promoter product was obtained by using the pJET-EX vector (SEQ ID NO: 9) as a template and forward and reverse primers (SEQ ID NOS: 17 and 18).

The left arm, the kanamycin resistance gene (Kan)(SEQ ID NO: 10), the tac promoter (Ptac)(SEQ ID NO: 1), and the right arm were inserted into the pMKO vector by using an IN-FUSION® GD cloning kit (Takara), thereby obtaining a pMKO-glycerol_Op_exp vector including left arm-Kan-Ptac-right arm.

Regarding the resultant vector, the left arm and the right arm are each homologous to a region for double crossover with respect to the promoter region of the glycerol operon of the genome, and are positioned at opposite ends of the resultant vector. Kan is a selection marker for identifying chromosome integration. The tac promoter was identified to constitutively induce expression in K. xylinus, and is used for overexpression or constitutive expression of the glycerol operon. The homologous recombination was confirmed by using genome DNA as a template and the primers of SEQ ID NOS: 19 and 20.

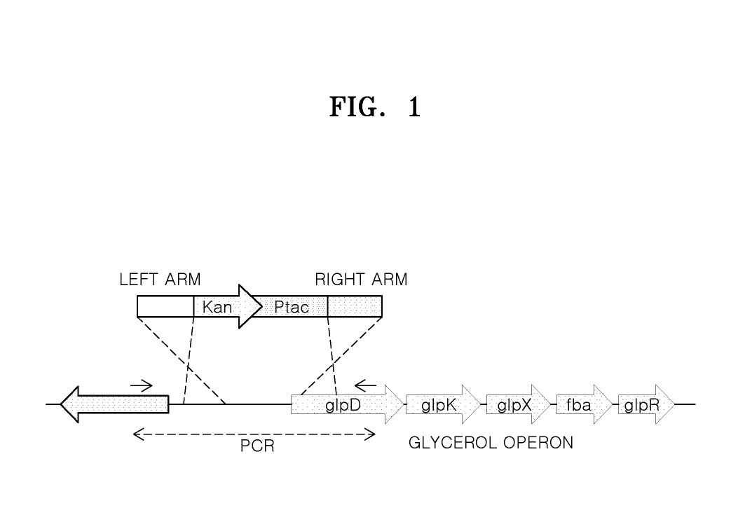

FIG. 1 illustrates a DNA construct for the replacement of the glycerol operon promoter, and a homologous recombination process.

(B) Transformation

The K. xylinus DSM 2325 strain was spread on a 2% glucose-added HS (Hestrin Schramm)-agar medium-containing plate, and then, cultured at a temperature of 30 □ for 3 days. The cultured strain was transferred to a 50 ml falcon tube by using sterilized water, and then, vortexed for 2 minutes. The 2% glucose-added HS-agar medium contained 0.5% peptone, 0.5% yeast extract, 0.27% Na₂HPO₄, 0.15% citric acid, 2% glucose, and 1.5% bacto agar. After 1% cellulase (Sigma, Cellulase from Trichoderma reesei ATCC 26921) was added thereto, the reaction proceeded at a temperature of 30 □ at 160 rpm for 2 hours, and then the result was washed with 1 mM HEPES buffer-containing medium, followed by washing with 15 (w/w) % glycerol three times and re-suspension with 1 ml 15 (w/w) % glycerol.

100 μl of the resultant competent cells was transferred to a 2 mm electro-cuvette, and then, 3 μg of the DNA construct was added thereto and electroporation (2.4 kV, 200 Ω, 25 μF) was performed thereon to perform transformation. The transformed cells were re-suspended in 1 ml 2% glucose-containing HS medium, then transferred to a 14 ml round-tube, then cultured at a temperature of 30 □ at 160 rpm for 2 hours, then spread on an HS-agar medium-containing plate supplemented with 2% glucose, 1 (v/v) % ethanol, and 5 μg/ml kanamycin, and then cultured at a temperature of 30 □ for 5 days.

PCR was carried out on colonies on the plate by using the primers of SEQ ID NOs: 19 and 20 to confirm that the DNA construct had been inserted into the chromosome. As a result, K. xylinus cells were obtained in which a promoter located at the 5′ end of the glycerol operon of genomic DNA was replaced by a tac promoter.

2. Confirmation of Glucose Consumption and Cellulose Production

The strain obtained as described in 1. (2) above was streaked on an HS-agar medium-containing plate supplemented with 2% glucose, 1% ethanol, and 5 μg/ml kanamycin, and then cultured at a temperature of 30 □ for 5 days. The cultured strain was inoculated into a 250 ml flask containing 50 ml of HS medium supplemented with 4% glucose and 1% ethanol, and cultured at 30° C. and 230 rpm for 5 days. As a result, cellulose (hereinafter referred to as “cellulose nanofiber (CNF)”) was produced on the surface of the medium directly in contact with air. The CNF was washed with 0.1N NaOH and distilled water at 60° C., and then freeze-dried to remove H₂O therefrom.

Glucose was analyzed by HPLC analysis using an Aminex HPX-87H column (Bio-Rad, USA). Table 1 shows CNF production and yield thereof of a K. xylinus strain in which a glycerol operon promoter was replaced with a tac promoter.

TABLE 1

| Strain | Glucose (g/L) | CNF production (g/L) | CNF yield (%) |
|---|---|---|---|
| Control | 5 | 1.0 | 19.0 |
| | 10 | 2.0 | 19.5 |
| | 20 | 2.8 | 13.9 |
| | 40 | 3.3 | 8.3 |
| Test group | 5 | 1.6 | 32.5 |
| Test group | 10 | 3.1 | 30.5 |
| | 20 | 3.8 | 19.0 |
| | 40 | 3.4 | 8.6 |

In Table 1, the control group was the K. xylinus DSM 2325 strain, and the test group was the K. xylinus strain into which the tac promoter was introduced. As shown in Table 1, the test group produced significantly more CNF than the control group. In detail, in the media containing 5, 10, 20, and 40 g/L of glucose, compared to the control group, the CNF production yields in the test group were increased by 70%, 56%, 36%, and 3%, respectively, and in the case of the CNF yields, 13.5%, 11.0%, 5.1% and 0.3%, respectively.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A recombinant microorganism comprising a genetic modification that enhances expression of at least one gene regulated by a glycerol operon selected from a gene encoding glycerol-3-phosphate dehydrogenase (glpD), a gene encoding glycerol kinase (glpK), a gene encoding fructose-1,6-bisphosphatase (glpX), and a gene encoding fructose-bisphosphate aldolase (FBA) 3.

2. The recombinant microorganism of claim 1, wherein the genetic modification comprises at least one modification selected from (i) a disruptive mutation of a regulatory element of the glycerol operon and (ii) substitution of an operator binding site or native promoter with a constitutive promoter.

3. The recombinant microorganism of claim 1, wherein the genetic modification is attenuation or inactivation of a glycerol-3-phosphate repressor.

4. The recombinant microorganism of claim 2, wherein the genetic modification is substitution of a promoter of the glycerol operon with a constitutive promoter.

5. The recombinant microorganism of claim 5, wherein the constitutive promoter is a tac promoter or a gap promoter.

6. The recombinant microorganism of claim 1, wherein the genetic modification increases the expression of the gene encoding fructose-1,6-bisphosphatase (glpX) and the gene encoding fructose-bisphosphate aldolase (FBA) 3.

7. The recombinant microorganism of claim 1, wherein the genetic modification increases a copy number of the at least one gene.

8. The recombinant microorganism of claim 1, wherein the glycerol-3-phosphate dehydrogenase (glpD) belongs to EC 1.1.5.3, EC 1.1.1.94, or EC 1.1.1.8, the glycerol kinase (glpK) belongs to EC 2.7.1.30, the fructose-1,6-bisphosphatase (glpX) belongs to EC 3.1.3.11, and the fructose-bisphosphate aldolase (FBA) 3 belongs to EC 4.1.2.13.

9. The recombinant microorganism of claim 1, wherein the glycerol-3-phosphate dehydrogenase (glpD), the glycerol kinase (glpK), the fructose-1,6-bisphosphatase (glpX), and the fructose-bisphosphate aldolase (FBA) 3 have a sequence identity of 85% or more with the amino acid sequences of SEQ ID NOS: 3, 4, 5, and 6, respectively.

10. The recombinant microorganism of claim 3, wherein the glycerol-3-phosphate regulon repressor (glpR) has a sequence identity of 85% or more with an amino acid sequence of SEQ ID NO: 7.

11. The recombinant microorganism of claim 1, wherein the microorganism is Komagataeibacter, Gluconacetobacter, or Acetobacter.

12. A method of producing cellulose, the method comprising: culturing the microorganism of claim 1 in a medium to produce cellulose, and collecting the cellulose from the culture.

13. The method of claim 12, wherein the medium comprises at least one selected from glucose and glycerol.

14. The method of claim 13, wherein a combined amount of the glucose and the glycerol is 20 g/L medium or less.

15. The method of claim 12, wherein the medium does not comprise glycerol.

16. The method of claim 12, wherein the genetic modification comprises at least one modification selected from (i) a disruptive mutation of a regulatory element of the glycerol operon and (ii) substitution of an operator binding site or native promoter with a constitutive promoter.

17. The method of claim 16, wherein the disruptive mutation is attenuation or inactivation of a glycerol-3-phosphate repressor.

18. The method of claim 17, wherein the substitution is substitution of a promoter of the glycerol operon with the constitutive promoter.

19. The method of claim 18, wherein the constitutive promoter is a tac promoter or a gap promoter.

20. The method of claim 12, wherein the genetic modification increases a copy number the at least one gene.

21. The method of claim 14, wherein the microorganism belongs to the genus Komagataeibacter, the genus Gluconacetobacter, or the genus Acetobacter.

22. A method of producing a microorganism having enhanced cellulose productivity, the method comprising introducing into a microorganism a genetic modification that increases the expression of at least one gene selected from a gene encoding glycerol-3-phosphate dehydrogenase (glpD), a gene encoding glycerol kinase (glpK), a gene encoding fructose-1,6-bisphosphatase (glpX), and a gene encoding fructose-bisphosphate aldolase (FBA) 3, wherein expression of the at least one gene is regulated by a glycerol operon, and the microorganism belongs to the genus Komagataeibacter, the genus Gluconacetobacter, or the genus Acetobacter.
